(19) United States
(12) Patent Application Publication
(10) Pub. No.: US 2011/0010782 A1
(43) Pub. Date: Jan. 13, 2011

(54) ADAPTING PARASITES TO COMBAT DISEASE

(75) Inventors: Eric J. Horvitz, Kirkland, WA (US); Simon John Mercer, Issaquah, WA (US); Sonia Patricia Carlson, Kirkland, WA (US); Chris Demetrios Karkanias, Sammamish, WA (US); David E. Heckerman, Bellevue, WA (US)

Correspondence Address: TUROCY & WATSON, LLP, 127 Public Square, 57th Floor, Key Tower, CLEVELAND, OH, 44114, US

(73) Assignee: MICROSOFT CORPORATION, Redmond, WA (US)

(21) Appl. No.: 12/500,115

(22) Filed: Jul. 9, 2009

(51) Int. Cl. A01K 67/00; G06F 19/00

(52) U.S. Cl. 800/8; 702/19

(57) ABSTRACT

Provided are systems and/or methods that facilitate sensing, detecting, logging, or treatment of a condition or need of a living body using a controlled parasite.

### BACKGROUND

Malaria is one of the world's major health problems. About one quarter of the world's population is exposed to the risk of malaria and more than a million people die of malaria each year. During the 20th century, economic and social development, together with anti-malarial campaigns, have resulted in the eradication of malaria from large areas of the world, reducing the affected area of the earth's land surface from 50% to 27%. Nonetheless, given expected population growth it is projected that by 2010 half of the world's population, nearly 3.5 billion people, will be living in areas where malaria is transmitted. Current estimates suggest that there are well in excess of 1 million deaths due to malaria every year, and the economic costs for Africa alone are staggering. These figures highlight the global malaria crisis and the challenges it poses to the international health community.

One of the most acute forms of the disease is caused by the protozoan parasite Plasmodium falciparum which is responsible for most of the mortality attributable to malaria. Another form of the disease is caused by Plasmodium vivax.

The life cycle of Plasmodium (e.g., P. falciparum or P. vivax) is complex requiring two hosts, man and mosquito, for completion. The infection of man is initiated by the inoculation of sporozoites in the saliva of an infected mosquito. The sporozoites migrate to the liver and there infect hepatocytes (liver stage) where they differentiate, via the exoerythrocytic intracellular stage, into the merozoite stage which infects red blood cells (RBC) to initiate cyclical replication in the asexual blood stage. The cycle is completed by the differentiation of a number of merozoites in the RBC into sexual stage gametocytes which are ingested by the mosquito, where they develop through a series of stages in the midgut to produce sporozoites which migrate to the salivary gland.

Individuals can be inoculated against malaria. Irradiated mosquitoes can be used to deliver damaged Plasmodium to individuals. Instead of contracting malaria, an individual receiving the damaged Plasmodium develops an immune response that renders the individual resistant to contracting malaria.

### SUMMARY

The following presents a simplified summary of the innovation in order to provide a basic understanding of some aspects described herein. This summary is not an extensive overview of the claimed subject matter. It is intended to neither identify key or critical elements of the claimed subject matter nor delineate the scope of the subject innovation. Its sole purpose is to present some concepts of the claimed subject matter in a simplified form as a prelude to the more detailed description that is presented later.

Electronics can be adapted to exploit novel methods of monitoring body functions and delivering useful substances to the body, including treatment of malaria and monitoring and/or treating other bodily conditions. By modifying or making a parasitic organism that can be programmatically controlled by a stimulus external to the altered parasitic organism, the parasitic organism can be a powerful tool in delivering therapeutic compounds. Control over the function of the parasitic organism permits the selective activation of a treatment protocol, since the altered parasitic organism has the inherent ability to conduct nanoscale manufacturing of one or more beneficial substances. Rather than build nanomachines from scratch, altered parasitic organisms can be exploited as custom designed nanomachines that manufacture and/or deliver useful substances or payloads to a host. In order to effectively manage the host's condition, the modified parasitic organism can be used to deliver beneficial care and/or to record the internal state of the host.

The following description and the annexed drawings set forth in detail certain illustrative aspects of the claimed subject matter. These aspects are indicative, however, of but a few of the various ways in which the principles of the innovation can be employed and the claimed subject matter is intended to include all such aspects and their equivalents. Other advantages and novel features of the claimed subject matter will become apparent from the following detailed description of the innovation when considered in conjunction with the drawings.

### BRIEF DESCRIPTION OF THE DRAWINGS

FIG. 1 illustrates a block diagram of an exemplary system that facilitates sensing, detecting, logging, or treatment of a condition of a living body using an engineered organism.

FIG. 2 illustrates an exemplary methodology for engineering an organism.

FIG. 2 illustrates an exemplary methodology for engineering an organism and monitoring the condition of the engineered organism.

FIG. 4 illustrates a block diagram of an exemplary system that facilitates sensing, detecting, logging, or treatment of a condition of a living body using an engineered organism, a controller and data storage.

FIG. 5 illustrates a block diagram of an exemplary system that facilitates sensing, detecting, logging, or treatment of a condition of a living body using an engineered organism and an analysis component.

FIG. 6 illustrates a block diagram of an exemplary system that facilitates sensing, detecting, logging, or treatment of a condition of a living body using an engineered organism using an intelligent agent component.

FIG. 7 illustrates a block diagram of an exemplary system that facilitates sensing, detecting, logging, or treatment of a condition of a living body using an engineered organism and alarms, logging, and/or querying.

### DETAILED DESCRIPTION

The claimed subject matter is described with reference to the drawings, wherein like reference numerals are used to refer to like elements throughout. In the following description, for purposes of explanation, numerous specific details are set forth in order to provide a thorough understanding of the subject innovation. It may be evident, however, that the claimed subject matter can be practiced without these specific details. In other instances, well-known structures and devices are shown in block diagram form in order to facilitate describing the subject innovation.

Various groups have pursued building a nanomachine capable of producing beneficial substances. However, a platform for one of the best (robust, efficient, etc.) nanomachines has already been built, namely parasitic organisms. The disclosure describes how to make and use altered parasitic organisms for beneficial results such as the treatment of disease. The systems described herein contain engineered organisms capable of producing and/or delivering fluids and/or biological materials. Fluids and biological materials can be any substance that can alter a physiological condition or a disease state within a living body. Typical fluids and biological materials affecting physiological conditions encompass nucleic acids, proteins and organic molecules. Parasites, as described herein, are complicated eukaryotic organisms equipped with cellular machinery to emit and/or uptake biologically active materials after proper genetic or environmental modification. While the innovations disclosed herein do not require the in situ manufacturing of biological materials by a parasite, parasites can be altered to produce specific nucleic acids and/or protein using genetic engineering techniques while organic molecules can be made through the presence of defined enzymatic pathways.

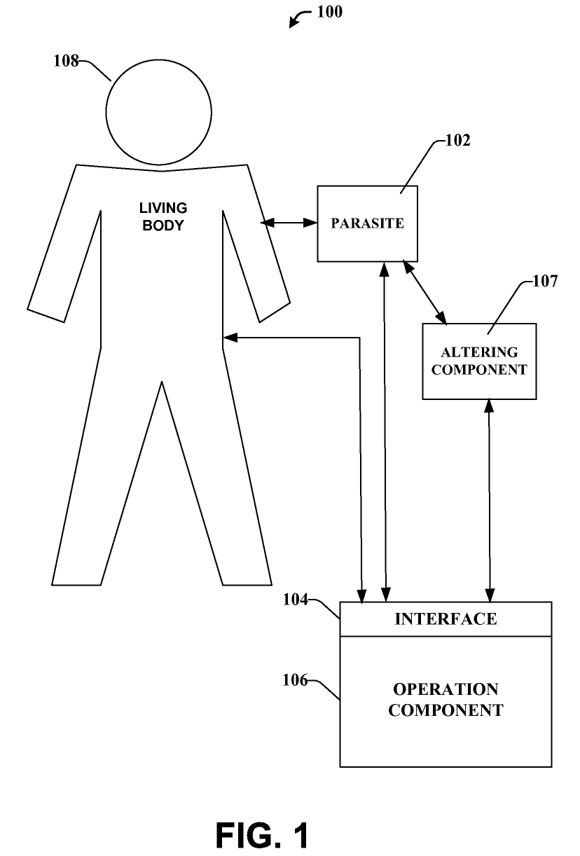

Turning now to the drawings, FIG. 1 illustrates a system 100 that facilitates exploiting a parasitic organism as a nanomachine for benefit of a living body. An altered parasitic organism 102 can provide a real-time treatment related to one or more particular conditions, wherein the condition can be associated with the living body and/or external factors affecting the living body. The altered parasitic organism 102 can be within or in contact with the living body 108 such that various conditions can be sensed, measured, detected, and/or treated, optionally in real-time. The altered parasitic organism 102 can also send/receive signals via the interface 104. An operation component 106 (or artificial processing center) can analyze the data concerning, received by, or generated by the altered parasitic organism 102 via an interface 104 (discussed infra). The operation component 106 can provide a plurality of analyses based at least in part upon the data related to a condition associated with the living body 108.

The altered parasitic organism 102 can be placed within the vicinity of the living body to provide treatment, wherein the operation component 106 can analyze such data to determine an efficient treatment regimen of the living body. The operation component 106 can provide treatments, diagnoses, recommendations, interventions, or other manipulations based at least in part upon the data generated by the altered parasitic organism 102.

An altered parasitic organism has at least one function or structure that is different from or unnatural compared to an unaltered parasite. In one embodiment, the altered function or structure of the altered parasitic organism is the ability to manufacture or express a protein or therapeutic molecule, typically an organic molecule. In another embodiment, the altered function or structure of the altered parasitic organism is the requirement to be periodically exposed to a specific chemical or environmental condition to maintain viability. In yet another embodiment, the altered function or structure of the altered parasitic organism is the ability to detect the presence of a specific protein, molecule, or environmental factor in a living body. The altered parasitic organism 102 is altered by an altering component 107 that affects some functional or physical change in a parasitic organism, wherein the new function or structure can be controlled and exploited to the benefit of a living body, such as a human being. Methods of altering parasitic organisms or changing the functionality thereof include one or more of genetic engineering techniques, ultrasound, radiation, chemical agents, compounds ingested (food), chemical shock, mutating agent, and the like.

In one embodiment, altered parasitic organisms can be made by genetically engineering parasites that naturally interact with a living body. The altered parasitic organisms sense, signal, provide and/or regulate metabolic substances or disease targeting substances. The subject innovation provides platforms for the programmatic treatment and detection of diseases and/or health deficiencies.

Genetically engineered refers to any method used to create an altered parasitic organism that expresses a protein or produces a compound or signal of interest, or exhibits sensitivity to a protein, compound, or environmental condition. Methods and vectors for genetically engineering parasites are known. Examples of genetic engineering techniques include expression vectors, targeted homologous recombination and gene activation, transactivation by engineered transcription factors, and the like.

Parasites that can be altered include human parasites and non-human parasites, such as animal parasites. Parasites are adept at interacting with living body hosts; such as contacting and exchanging fluids or substances with living body hosts or by penetrating into and residing in living body hosts. Examples of substances that can be exchanged between a living body and an altered parasitic organism include blood, saliva, bacteria, viruses, proteins, a pharmaceutical, a vitamin or co-factor, a vaccine, a genetically modified organism, and the like. Owing to the natural affinity of parasites to specific areas of a living body, altered parasitic organisms can be advantageous in situations where a specific organ or tissue of the living body is targeted for treatment. When possible, it is helpful to mitigate the pathogenic affects of the parasite, if any.

Specific examples of alterable parasitic organisms include mosquitoes, fleas, ticks, bed bugs (Cimicidae Cimex lectularius), midges (such as Ceratopogonidae), other blood sucking arthropods, annelids or leeches, nematodes such as Ascaris lumbricoides (roundworm which typically invades the gastrointestinal tract and lungs), pinworms such as Enterobius vermicularis (gastrointestinal tract, colon, fingertips), whipworms such as Trichuris trichiuria (gastrointestinal tract), flukes or trematodes such as Fasciola hepatica, Fasciolopsis buski (intestinal fluke) and schistosomes (liver and gallbladder), tapeworms or cestodes such as those from the genus Taenia (gastrointestinal tract), hookworms, heart worms, roundworms, lice (head, body, and pubic), and the like.

Altered parasitic organisms can be made using known techniques. For example, recombinant DNA techniques can be employed to make a genetically engineered mosquito having a desired function. Recombinant DNA is typically in the form of a vector. The vector can, for example, be a plasmid, cosmid, phage, or artificial chromosome. Vectors frequently include one or more selectable markers to enable selection of cells transformed (or transfected: the terms are used interchangeably in this specification) with them and, preferably, to enable selection of cells harboring vectors incorporating heterologous DNA. Appropriate “start” and “stop” signals are typically present. Additionally, if the vector is intended for expression, sufficient regulatory sequences to drive expression are present. Cloning vectors can be introduced into friendly bacteria, E. coli, or another suitable host which facilitates their manipulation.

Nucleic acid sequences for making the altered parasitic organism can be prepared by any convenient method involving coupling together successive nucleotides, and/or ligating oligo- and/or poly-nucleotides, including cell-free in vitro processes, but recombinant DNA technology forms the method of choice. Ultimately, nucleic acid sequences can be introduced into host cells by any suitable means.

The genome of the unaltered and altered parasitic organisms can be programmatically represented, and the interface 104 as directed by the operation component 106 can compute and program areas of modification for the altering component 107. For example, the interface 104 can direct altering component 107 to apply specific type/wavelength of radiation to specific area of the genome to turn “on”/“off” certain characteristics of the genome. Use of a program to target selected regions using specific radiation, then altering a parasite to have a functional change.

Furthermore, the system 100 can include any suitable and/or necessary interface component 104 (herein referred to as “interface 104”), which provides various adapters, collectors, connectors, channels, communication paths, receivers, etc. to integrate the operation component 106 into virtually any operating and/or database system(s). The interface 104 can receive data, wherein the data received can relate to the altered parasitic organism 102; the altering component 107; the living body 108; data associated with the living body; systems associated with the living body 108; collected data about a condition; any suitable data related to a living body condition, etc. In addition, the interface component 104 can provide various adapters, collectors, connectors, channels, communication paths, receivers, etc. that provide for interaction with the operation component 106. The results of the sensed information can be used to direct future analysis and/or altered parasitic organism operation.

The interface 104 can be used to send signals to or receive signals from the altered parasitic organism 102 and/or the altering component 107. These signals enable the altered parasitic organism 102 to function as a treatment vehicle or an active sensor. Alternatively, these signals enable the operation component 106 to access real time information concerning the living body 108 that can improve ongoing or future treatments. By way of illustration, the interface 104 can receive data or directly detect a compound, nucleic acid or protein produced by the parasitic organism in response to a chemical or environmental condition within the living body, or the interface can receive data or directly detect a protein, nucleic acid or therapeutic molecule manufactured by the parasitic organism for delivery to the living body to monitor the population level and/or effectiveness of the parasitic organism. The interface 104 can also receive data or directly detect for the presence of proteins, nucleic acids or molecules sampled from the living body for later analysis in cases where the parasitic organism is located external to the living body or is excreted or removable from the living body, e.g., feces and/or blood sample.

The programmatic interface 104 in cooperation with the operation component 106 can define where to target (what aspect, tissue, or specific location of the living body 108 to target), mode required to hit target (define the transport pathway to deliver a treatment to the target), if/how to monitor and log changes effected by the altered parasite (recording changes allows one to access the efficacy of a given treatment module).

It is to be appreciated that the system 100 can include a plurality of altered parasitic organisms 102 and the system 100 depicted is not to be limiting. For instance, the system 100 can include a plurality of altered parasitic organisms 102 with a single interface 104; multiple altered parasitic organisms 102 with multiple interfaces 104 and a plurality of operation components 106; etc. In other words, the system 100 can utilize any suitable number of altered parasitic organisms, interfaces, and/or sensor operation components to treat, track, monitor, collect, and/or record, data associated with the living body 108. Moreover, it is to be appreciated that the interface component 104 and the operation component 106 can be stand-alone components and not embedded in the living body environment.

In order to control the population of altered parasites (to avoid runaway breeding that can allow altered parasites to revert to their purely parasitic action), an activation and/or termination function for the altered parasite can be included. The altered parasites can be designed to mitigate harmful effects traditionally associated with the parasite (such as a mosquito capable of destroying or disabling malarial parasites therein), in order to deliver only the payloads desired. For example, a termination feature can include programmed death or reduced viability, unless a compound is delivered to the altered parasites (for example, a mosquito must ingest a certain compound every time period in order to remain alive or to reproduce). Furthermore, a termination signal can be sent by the interface component 104 to cause death of the altered parasite 102, or the altered parasite 102 can send the termination signal just before death to inform the operation component 106 that the altered parasite 102 is ceasing to operate.

Any signal sent by the altered parasite 102 can be in the form of a protein, nucleic acid, or molecule produced by the altered parasite 102 in response to a certain event, environmental condition, or upon happening of a binding event involving a receptor on the altered parasite. In situations where the parasitic organism is external to the living body, the termination signal transmitted to the altered parasite 102 can be the removal of a food source, introduction of a toxin or repellant into the food source, removal of a required nutrient or chemical compound from the food source or environment necessary for the parasitic organism's viability, or introduction of a protein, nucleic acid or molecule that induces programmed death or reduced viability in the altered parasite 102. In situations where the parasitic organism is internal to the living body, the termination signal can be the removal of a protein or chemical compound necessary for the parasitic organism's viability that is administered to the living body or the introduction of a chemical compound or protein to the living body that results in death of the altered parasite 106.

The operation component 106 can also have the ability to send an activation signal to the altered parasite 102. The activation signal can be a protein, nucleic acid or molecule placed in the food supply, into the environment or administered to the living body in the case of an internal parasite. In one embodiment, the protein, nucleic acid or molecule acting as an activation signal binds to a receptor on the altered parasite 102 to allow for production of a protein, nucleic acid or therapeutic molecule by the altered parasite 102. In another embodiment, the protein, nucleic acid or molecule acting as an activation signal is placed in the food supply, where the activation signal repels wild-type parasites found in the environment, while the altered parasite 102 in not repelled and is able to access the food supply that can contain a fluid or biological material for delivery to the living body.

Mutation rates (altered parasites that do not contain the desired characteristics) can be controlled by applying a screening feature to programmatic control by the interface component 104 and the operation component 106. The screening feature increases the effectiveness of the termination feature. The screening feature can alternatively involve detecting a target region of the genome to determine if a desired change is effected.

Mutations can advantageously be examined and studied before mutants are destroyed to determine if beneficial mutations exist. Beneficial mutations can be exploited by further breeding and application to further uses. New functionalities can be recorded and logged for future use.

Programmed behavior can include a location or destination seeking feature that can facilitate collection and study (or exit plan).

Altered parasites that deviate from the desired features can be programmed to exhibit a characteristic that facilitates screening; such as fluoresce, grow big wings to be caught by a physical filter, etc. Alternatively, mutants (altered parasites that do not contain the desired characteristics) express a “screening gene” to manifest fluoresce, grow big wings, etc.

An example of system 100 includes the use of a mosquito as the parasite. The mosquito is genetically modified so that when a Plasmodium protozoan is ingested, the mosquito breaks down or inactivates the Plasmodium protozoan. Alternatively, the mosquitoes carrying the Plasmodium protozoan are modified with specific wavelength radiation so that the Plasmodium protozoan is inactivated. Still alternatively, the mosquitoes carrying the Plasmodium protozoan are modified with ultrasound with a resonance frequency of Plasmodium protozoan thereby destroying the Plasmodium protozoan. In any of the three scenarios, the mosquito is then used as a delivery device to deliver a payload of inactive Plasmodium protozoan that can induce an immune response in a living body against malaria. The altering components of the genetically modifications, specific wavelength radiation, or ultrasound with a resonance frequency include transcription techniques, an irradiating device, or an ultrasound device, respectively. Any of the genetically modifications, specific wavelength radiation, or ultrasound with a resonance frequency are directed by interface 104 coupled to an operation component 106.

Due to the mosquitoes' natural affinity to penetrate the human skin, upon biting a human, the mosquito delivers the desired payload of an inactive Plasmodium protozoan, analogous to a malaria vaccine. Alternatively or additionally, if the mosquitoes provided feeding troughs with a nutrient and/or medicine, as determined programmatically by an operation component, the nutrient and/or medicine can also be provided to the living body.

Although the mosquitoes serve as vector to deliver inactive Plasmodium protozoan, there is a possibility for the mosquitoes to pick up active Plasmodium protozoan or other harmful agents from the environment. To guard against this possibility, the mosquitoes incorporate a termination feature wherein the mosquitoes can be controlled after released and/or killed if the need arises.

Another example of a functional change of an altered parasitic organism, such as fluke, includes an organism that releases insulin (in essence, an altered parasitic organism acting a pancreatic cell) in a host or living body colonized by the altered parasitic organism. The fluke is genetically modified so that when the glucose level is above a predetermined threshold, insulin can be released (and only when blood glucose levels exceed a predetermined threshold). That is, if blood glucose rises above a certain level, insulin can be manufactured and released by the organism. If blood glucose falls below a certain level insulin manufacture and release by the organism can be stopped. However, unlike the pancreas, the altered parasitic organism can be turned “on” and/or “off” through the use of an initiation or termination signal.

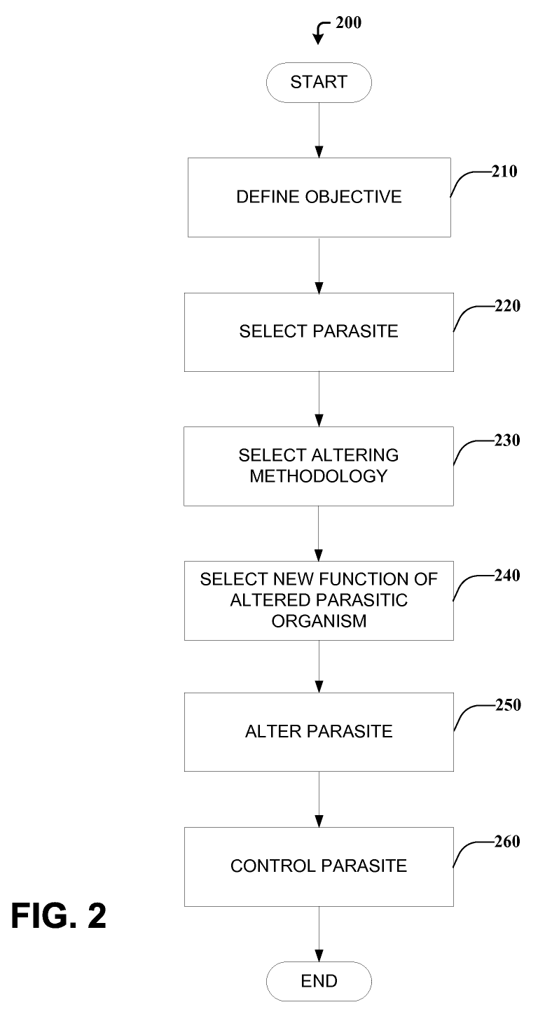

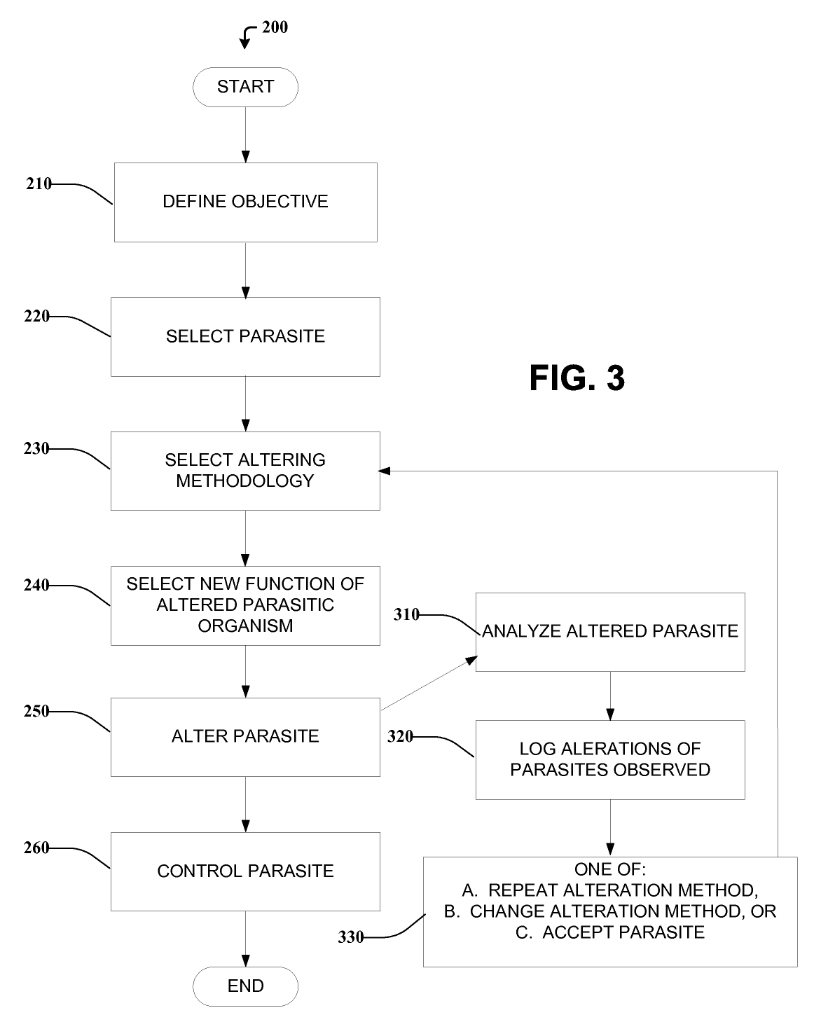

The operation component 106 considers each fixed and desired attribute of the system and implements selections and acts to achieve a given objective. The capabilities of the operation component 106 can be illustrated the method 200 depicted in FIG. 2.

In act 210, a given objective is identified. Examples of an objective include identifying a disease or human condition that requires treatment, a disease or human condition that can be detected, a disease, physical condition or human condition that can be acknowledged through a signal, or even identifying a desired treatment. There are typically a number of factors and attributes associated with a given disease or human condition that impact selection of parasite, altering methodology, and altered parasite function. Such factors and attributes are considered and weighted in the following acts.

In act 220, a parasite is selected to undergo an alteration to provide an altered parasitic organism. The parasitic organism is selected so that the location or nature of the disease or human condition is some way related to the natural function of the parasite. For example, a condition in the small intestine raises the likelihood that a tapeworm can be a suitable candidate for the altered parasitic organism. Other factors such as duration of the treatment and the manner in which the treatment can be delivered, can also be considered when selecting the parasite.

In act 230, the manner in which the selected parasite is altered is selected. The selection of altering methodology is dependent upon a number of factors including the identity of the parasite, the intended new function of the altered parasitic organism, the desired number of new functionalities, and the like. Often, two or more altering methodologies are employed to provide the altered parasitic organism. In the event that the altering methodology is genetic engineering, additional operational selections can be made including selecting a vector, host organism, mode of transcription, etc.

In act 240, the desired function of the altered parasitic organism is selected. For example, the desired function can include delivering a payload (of a substance beneficial to the living body or detrimental to a disease or unhealthy condition), sensing a condition that can trigger another function (when a certain substance exceeds or falls below a given threshold), detecting for the presence of a disease or unhealthy condition, instituting self induced parasite death, and the like.

In act 250, the altering component is engaged to alter the selected parasitic organism in the manner proposed by the operation component. The altering component can additionally treat the parasite so as to mitigate the natural and inherent characteristics of the parasite. For example, if the parasite is a mosquito, in addition to whatever new beneficial function is bestowed on the altered mosquito, the mosquito can be treated so any bacteria therein are destroyed thereby mitigating the risk that a harmful disease such as malaria is unintentionally passed along to a living body undergoing treatment in accordance with the methods described herein.

In act 260, in some instances, the operation component triggers the altered parasitic organism (if the altered parasitic organism does not do so naturally) to begin treatment of the living body.

As shown in method 300 in FIG. 3, the operation component 106 can perform additional optional functions. As discussed above, altered parasites that deviate from the desired features can be programmed to exhibit a characteristic that facilitates screening. In act 310, the altered parasites are analyzed for a programmed characteristic or subjected to a biochemical analysis to determine the nature and extent of any alteration introduced in act 250. In act 320, the observance of any observed observation is logged. It is feasible for the development of a desired altered parasite to be achieved through an iterative process, wherein multiple rounds of alteration (act 250) may be required to achieve the desired results. Further, it is feasible that more than one type of alteration may be required to develop the desired features in the altered parasite. In act 330, the alterations analyzed in act 340 are evaluated to determine the next appropriate action. It can be determined that the alteration method selected in act 230 needs to be repeated, that a different alteration method is needed, or that the alteration has been performed satisfactory manner. In particular, acts 310, 320 and 330 can be performed to further develop a termination feature of the altered parasite. However, it is understood that acts 310, 320, and 330 can be directed toward the development of any objection identified in act 210 in accordance with the innovations disclosed herein.

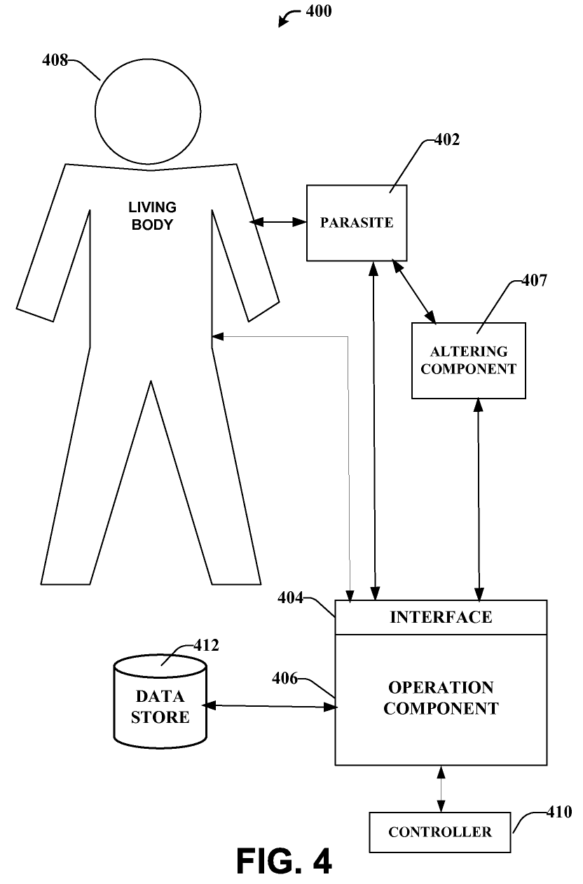

FIG. 4 illustrates a system 400 that facilitates treating characteristics associated with a living body that utilizes an altered parasitic organism. The system 400 includes a living body 408 and an altered parasitic organism 402 that receives data via an interface 404. The altered parasitic organism 402 deliver a payload to address symptoms associated with a disease or condition of the living body 408 to provide improved health quality. Furthermore, an operation component 406 can provide analysis, processing, etc. based at least in part upon the data of a treatment regimen or generated by the altered parasitic organism 402. It is to be appreciated that the altered parasitic organism 402, the living body 408, and the operation component 406 can be substantially similar to the altered parasitic organism 402, the living body 408, and the operation component 406 of FIG. 1, respectively. Although a single altered parasitic organism 402 is illustrated in the system 400, it is to be appreciated and understood that the claimed subject matter is not so limited and any number of altered parasitic organisms can be utilized. The multiple altered parasitic organisms can operate by exchanging data and/or analysis between each other or the operation component.

The in situ data collected, detected, generated, signaled and/or monitored data related to the living body 408 can be analyzed and/or processed by the operation component 406. Furthermore, the operation component 406 can provide data manipulations based at least in part upon the data collected, generated, detected, and/or monitored by the symbiotic agent 402 within the living body 408. Moreover, the data collected, generated detected, and/or monitored can be utilized by a controller 410 coupled to the operation component 406. In accordance with an aspect of the subject innovation, the controller 410 can be a programmable logic controller (PLC). While PLCs can be utilized within the system 400 as the controller 410, it is to be understood that any suitable automation controller can be employed in connection with the claimed subject matter. For example, any suitable microprocessor and/or microcontroller can be utilized within the system 400 as the controller 410 including a personal computer or single-board computer with suitable input-output circuitry. Moreover, it is to be appreciated that the controller 410 can contain software components and hardware components having inputs and/or outputs that can be utilized in connection with analyzing or identifying conditions of the living body.

The operation component 406 can utilize a data store 412, wherein the data store 412 can store various data related to the system 400. The data store 412 can provide storage for in situ data collected via the parasite 402, configurations, conditions, health standards, control signal, system response, quality, disease/illness information, profiles, historic data, calibration data, security data, safety data, etc. The data store 412 can be, for example, either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable programmable ROM (EEPROM), or flash memory. Volatile memory can include random access memory (RAM), which acts as external cache memory. By way of illustration and not limitation, RAM is available in many forms such as static RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), Rambus direct RAM (RDRAM), direct Rambus dynamic RAM (DRDRAM), and Rambus dynamic RAM (RDRAM). The data store 412 of the subject systems and methods is intended to comprise, without being limited to, these and any other suitable types of memory. In addition, it is to be appreciated that the data store 412 can be a server, a database, a hard drive, and the like.

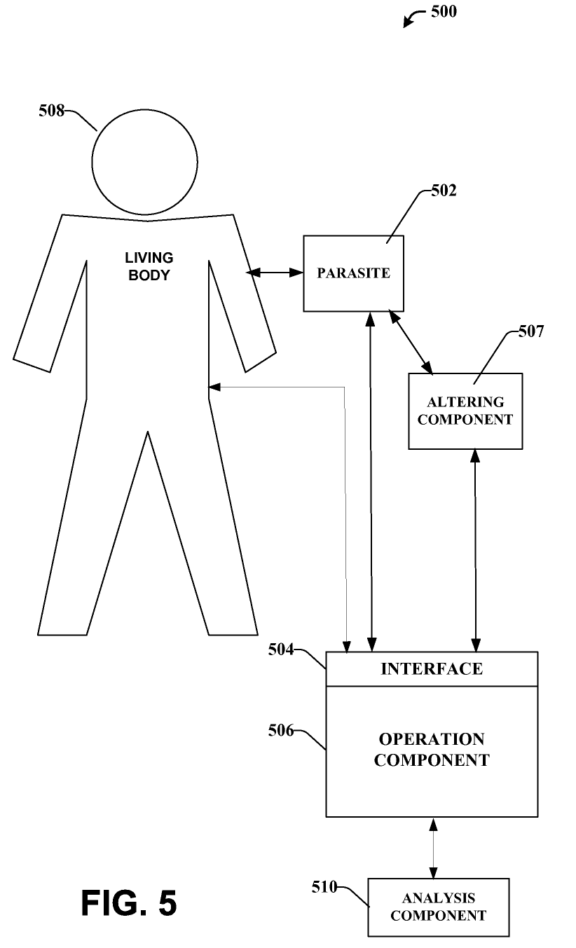

FIG. 5 illustrates a system 500 that facilitates that facilitates treating characteristics associated with a living body that utilizes an altered parasitic organism. The system 500 includes a living body 508 and an altered parasitic organism 502 that receives or sends data via an interface 504. The altered parasitic organism 502 delivers a payload to address symptoms associated with a disease or condition of the living body 504 to provide improved health quality. The system 500 can include a parasitic organism 502 that provides continuous sensing, monitoring and/or detection in a living body 508. It is to be appreciated that the parasite 502, operation component 506, and the analysis component 510 can be substantially similar to previously described components. The analysis component 510 can analyze data generated by the parasite 502 in order to identify an illness or disease or to determine the appropriate level of therapeutic agent (fluid or biological material) to administer. For example, the parasite 502 can include a number of different receptors specific for various different maladies. The parasite 502 signals the operation component 506 via the interface 504 with data that can be extrapolated to determine which receptor is occupied, and what the significance of the bound receptor is. Alternatively, the parasite 502 produces a signal compound indicating to the operation component 506 via the interface 504 that a certain receptor is experiencing a binding event. Further, the parasitic 502 can sample fluids from the living body to be analyzed at a later time where the parasitic organism 502 is excreted from the body or retrievable through a blood sample taken from the living body. Although the interface 504 is shown directly coupled with the operation component 506, the interface 504 can be directly coupled with the living body 508, such as a device held within a wrist band, ankle band, clothing article, shoe, etc.

The analysis component 510 can contain a classifier. A classifier is a function that maps an input attribute vector, x=(x1, x2, x3, x4, xn), to a confidence that the input belongs to a class, that is, f(x)=confidence(class). Such classification can employ a probabilistic and/or statistical-based analysis and can further be influenced by a consideration of utilities or costs associated with correct and incorrect classification to prognose or infer an action that a user desires to be automatically performed. A support vector machine (SVM) is an example of a classifier that can be employed. The SVM operates by finding a hypersurface in the space of possible inputs, where the hypersurface splits the triggering criteria from the non-triggering events. Intuitively, this makes the classification correct for testing data that is near, but not identical to training data. Other model classification approaches include, e.g., naive Bayes, Bayesian networks, decision trees, neural networks, fuzzy logic models, and probabilistic classification models assuming different patterns of independence can be employed. Classification as used herein also is inclusive of statistical regression that is utilized to develop models of priority.

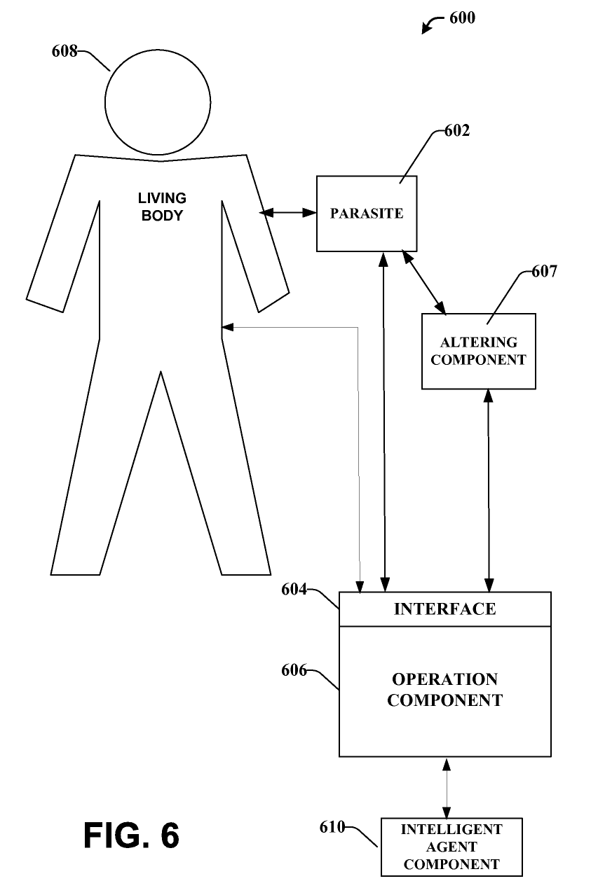

FIG. 6 illustrates a system 600 that facilitates monitoring a living body with symbiotic agent in real-time. The system 600 can include an altered parasite 602 that can be incorporated into a living body 603. An operation component 606 can provide data manipulations based at least in part upon the collected real-time data, wherein such manipulations can provide treatment, diagnosis, prognosis, control, detection, of a condition of the living body 608.

The system 600 can include an intelligent agent component 610 that can be deployed on commercially available PLC's, PC's, SBC, etc. within or connected to the operation component 606. It is to be appreciated that the claimed subject matter can include multiple altered parasites 602 and/or multiple intelligent agent components 610. Moreover, it is to be understood that the intelligent agent component 610 can be a stand-alone component or incorporated into the operation component 606. The intelligent agent component 610 can provide holonic system capabilities and the eventual transition to autonomous agents, which can respond to unexpected detections/data, the ability to dynamically respond to the unexpected detections/data. The intelligent agent component 608 can allow highly distributed diagnostics to sense an unfavorable condition and prescribe a superior countermeasure.

The intelligent agent component 610 can utilize a suite of simulation, prototyping, and/or deployment tools in accordance with the subject innovation. By deploying the intelligent agent component 610, unprecedented intervention can be provided as well as consequent overall health benefits. The system 600 can significantly enhance the quality, health, and efficacy of living bodies. Furthermore, the real-time process information can be utilized for closed-loop feedback control, adaptive process model development, predictive treatment, and other intervention applications.

The system 600 can enable the living body to function during illness, deficiency, or disease. Moreover, the intelligent agent component 610 can adhere to published agent-to-agent communication protocols (e.g., Foundation for Intelligent Physical Agents (FIPA)) and provide local intervention and decision-making along with more overall health goals.

It is to be understood that the intelligent agent component 610 can provide for reasoning about or infer states of the system, environment, and/or user from a set of observations as captured via events and/or data. Inference can be employed to identify a specific context or action, or can generate a probability distribution over states, for example. The inference can be probabilistic—that is, the computation of a probability distribution over states of interest based on a consideration of data and events. Inference can also refer to techniques employed for composing higher-level events from a set of events and/or data. Such inference results in the construction of new events or actions from a set of observed events and/or stored event data, whether or not the events are correlated in close temporal proximity, and whether the events and data come from one or several event and data sources. Various classification (explicitly and/or implicitly trained) schemes and/or systems (e.g., support vector machines, neural networks, expert systems, Bayesian belief networks, fuzzy logic, data fusion engines, etc.) can be employed in connection with performing automatic and/or inferred action in connection with the claimed subject matter.

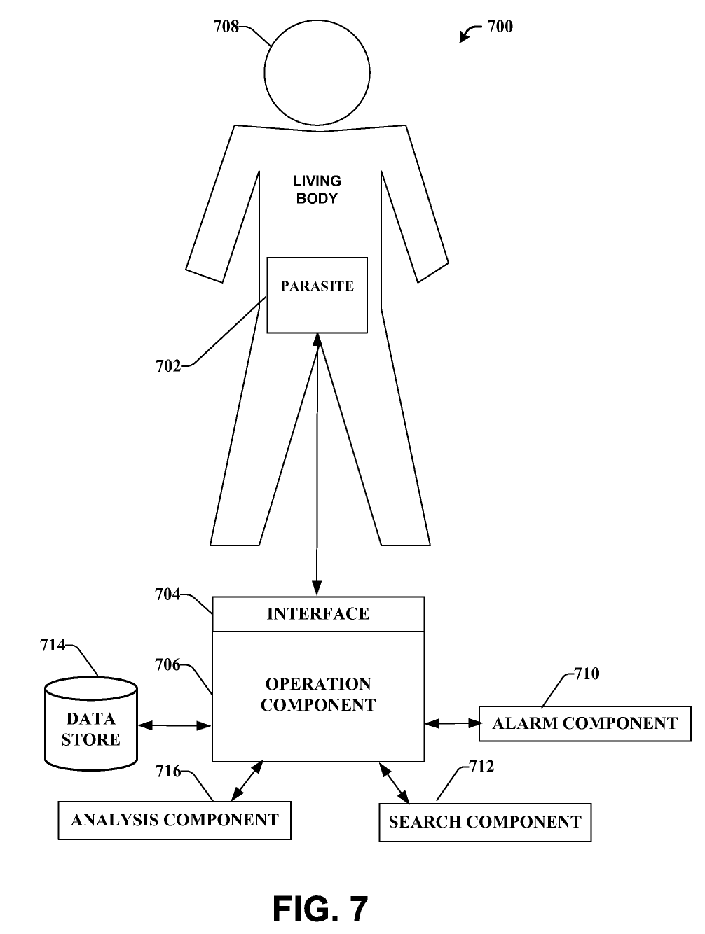

FIG. 7 illustrates a system 700 that facilitates implementing altered parasites within a living body 708 providing alarms, data logging/searching, event logging, and/or querying. The system 700 can include an altered parasite 702 that provides monitoring, detection, and/or data collection within a living body 708. The collected data can be utilized by an operation component 706, wherein the data can be implemented for analysis, computations, determinations, and/or any other manipulations based upon the collected data.

The operation component 706 can utilize an alarm component 710 that can provide alarms and/or warnings associated with data generated by the symbiotic agent based at least in part upon the health severity of the measurements, detections, and/or data collections via the altered parasite 702 and/or historical/nominal data. For example, the alarm component 710 can implement an alarm or unambiguous warning such as, warning lights, pop-up screens, blinking data display items, graphical items, email, text, cellular communication, web site activity, etc. when a particular condition is detected or measured beyond a specific threshold. It is to be appreciated that the status and/or alarms can be stored in the data store 714 (described previously), a “black box” recorder, etc. Additionally, the alarm component can signal a condition that can occur in the future to permit action to be taken before a condition occurs or before actions causing a condition are performed.

The operation component 706 can further utilize a search component 712 that allows querying of the system 700. In particular, the search component 712 can provide querying of any data collected or generated by the symbiotic agent 702, stored data, system data, conditions of the living body 708, analytical data, historical data and/or any other data related to the system 700. For instance, the search component 612 can be utilized to discover data related to a particular condition and circumstances present when the condition was measured/detected. It is to be appreciated that although the search component 712 is illustrated as a stand-alone component, the search component 712 can be incorporated into the operation component 706. It is to be appreciated that the system components can be distributed and remote from the living body 708 and remote from each other such as accessible via Internet, Ethernet, etc.

In addition, the search component 712 can further employ a log component wherein the log component can store and/or track various data related to the system 700. The system 700 can include an analysis component 716, which can analyze the significance of data generated by the altered parasite 702, and prescribed suitable countermeasures to conditions detected within the living body 708. Data generated by modified parasitic organisms can be collected and analyzed, providing powerful information to devise new and alternative treatments and interventions for diseases, deficiencies, preventions, and so forth.

It is to be appreciated and understood that directions in synthetic biology are enabling increasingly flexible and reusable specifications of systems based on inventories of well-characterized components and assemblies such as channels, timers, switches, etc. Moreover, introduced biological systems can employ components that record and/or remember multiple states over time, as encoded in a single and/or multiple molecules.

As utilized herein, terms “component,” “system,” “interface,” “device,” “generator,” “collector,” and the like are intended to refer to a computer-related entity, either hardware, software (e.g., in execution), and/or firmware. For example, a component can be a process running on a processor, a processor, an object, an executable, a program, a function, a library, a subroutine, and/or a computer or a combination of software and hardware. By way of illustration, both an application running on a server and the server can be a component. One or more components can reside within a process and a component can be localized on one computer and/or distributed between two or more computers.

Furthermore, the claimed subject matter can be implemented as a method, apparatus, or article of manufacture using standard programming and/or engineering techniques to produce software, firmware, hardware, or any combination thereof to control a computer to implement the disclosed subject matter. The term “article of manufacture” as used herein is intended to encompass a computer program accessible from any computer-readable device, carrier, or media. For example, computer readable media can include but are not limited to magnetic storage devices (e.g., hard disk, floppy disk, magnetic strips, etc.), optical disks (e.g., compact disk (CD), digital versatile disk (DVD), etc.), smart cards, and flash memory devices (e.g., card, stick, key drive, etc.). Additionally it should be appreciated that a carrier wave can be employed to carry computer-readable electronic data such as those used in transmitting and receiving electronic mail or in accessing a network such as the Internet or a local area network (LAN). Of course, those skilled in the art will recognize many modifications can be made to this configuration without departing from the scope or spirit of the claimed subject matter. Moreover, the word “exemplary” is used herein to mean serving as an example, instance, or illustration. Any aspect or design described herein as “exemplary” is not necessarily to be construed as preferred or advantageous over other aspects or designs.

While the claimed subject matter has been described above in the general context of computer-executable instructions of a computer program that runs on a local computer and/or remote computer, those skilled in the art will recognize that the subject innovation can also be implemented in combination with other program modules. Generally, program modules include routines, programs, components, data structures, etc., that perform particular tasks and/or implement particular abstract data types.

Moreover, those skilled in the art will appreciate that the inventive methods can be practiced with other computer system configurations, including single-processor or multi-processor computer systems, minicomputers, mainframe computers, as well as personal computers, hand-held computing devices, microprocessor-based and/or programmable consumer electronics, and the like, each of which can operatively communicate with one or more associated devices. The illustrated aspects of the claimed subject matter can also be practiced in distributed computing environments where certain tasks are performed by remote processing devices that are linked through a communications network. However, some, if not all, aspects of the subject innovation can be practiced on stand-alone computers. In a distributed computing environment, program modules can be located in local and/or remote memory storage devices.

What has been described above includes examples of the subject innovation. It is, of course, not possible to describe every conceivable combination of components or methodologies for purposes of describing the claimed subject matter, but one of ordinary skill in the art will recognize that many further combinations and permutations of the subject innovation are possible. Accordingly, the claimed subject matter is intended to embrace all such alterations, modifications, and variations that fall within the spirit and scope of the appended claims.

In particular and in regard to the various functions performed by the above described components, devices, circuits, systems and the like, the terms (including a reference to a “means”) used to describe such components are intended to correspond, unless otherwise indicated, to any component which performs the specified function of the described component (e.g., a functional equivalent), even though not structurally equivalent to the disclosed structure, which performs the function in the herein illustrated exemplary aspects of the claimed subject matter. In this regard, it will also be recognized that the innovation includes a system as well as a computer-readable medium having computer-executable instructions for performing the acts and/or events of the various methods of the claimed subject matter.

There are multiple ways of implementing the present innovation, e.g., an appropriate API, tool kit, driver code, operating system, control, standalone or downloadable software object, etc. which enables applications and services to use the techniques of the invention. The claimed subject matter contemplates the use from the standpoint of an API (or other software object), as well as from a software or hardware object that operates according to the techniques in accordance with the invention. Thus, various implementations of the innovation described herein can have aspects that are wholly in hardware, partly in hardware and partly in software, as well as in software.

The aforementioned systems have been described with respect to interaction between several components. It can be appreciated that such systems and components can include those components or specified sub-components, some of the specified components or sub-components, and/or additional components, and according to various permutations and combinations of the foregoing. Sub-components can also be implemented as components communicatively coupled to other components rather than included within parent components (hierarchical). Additionally, it should be noted that one or more components can be combined into a single component providing aggregate functionality or divided into several separate sub-components, and any one or more middle layers, such as a management layer, can be provided to communicatively couple to such sub-components in order to provide integrated functionality. Any components described herein can also interact with one or more other components not specifically described herein but generally known by those of skill in the art.

In addition, while a particular feature of the subject innovation may have been disclosed with respect to only one of several implementations, such feature can be combined with one or more other features of the other implementations as may be desired and advantageous for any given or particular application. Furthermore, to the extent that the terms “includes,” “including,” “has,” “contains,” variants thereof, and other similar words are used in either the detailed description or the claims, these terms are intended to be inclusive in a manner similar to the term “comprising” as an open transition word without precluding any additional or other elements.

What is claimed is:

1. A system that facilitates delivery of a fluid or biological material to a living body, comprising:
an altered parasitic organism, wherein an alteration to the altered parasitic organism includes at least a termination function capable of receiving a termination signal and wherein the termination signal results in the death or reduced viability of the altered parasitic organism;
an interface component that facilitates delivery of the termination signal and facilitates receipt of or delivery of data associated with the condition of the living body; and
an operation component that analyzes received data associated with the condition of the living body or provides data delivery to the altered parasitic organism.

2. The system of claim 1, the living body comprising a human being.

3. The system of claim 1, the altered parasitic organism is one species selected from the group comprising: mosquitoes, fleas, ticks, bed bugs (Cimicidae Cimex lectularius), midges (such as Ceratopogonidae), other blood sucking arthropods, annelids or leeches, nematodes such as Ascaris lumbricoides (roundworm which typically invades the gastrointestinal tract and lungs), pinworms such as Enterobius vermicularis (gastrointestinal tract, colon, fingertips), whipworms such as Trichuris trichiuria (gastrointestinal tract), flukes or trematodes such as Fasciola hepatica, Fasciolopsis buski (intestinal fluke) and schistosomes (liver and gallbladder), tapeworms or cestodes such as those from the genus Taenia (gastrointestinal tract), hookworms, heart worms, roundworms, lice (head, body, and pubic), and the like.

4. The system of claim 1, the altered parasitic organism comprising a mosquito containing Plasmodium falciparum in an active or inactive state.

5. The system of claim 1, wherein the interface performs one or more of receiving data or directly detecting a compound, nucleic acid or protein produced by the parasitic organism in response to a detected chemical or environmental condition within the living body and receiving data or directly detecting a protein, nucleic acid or therapeutic molecule manufactured by the parasitic organism for delivery to the living body.

6. The system of claim 1, wherein the altered parasitic organism has an initiation function capable of receiving an initiation signal and wherein the initiation signal results in the altered parasitic organism manufacturing a fluid or biological material or allows the altered parasitic organism to receive a fluid or biological material for delivery to the living body.

7. The system of claim 1, wherein receipt of or delivery of data associated with the condition of the living body comprises data indicative of a receptor binding event on the altered parasitic organism.

8. A method of delivering a fluid or biological material to a living body, comprising:
identifying a fluid or biological material for delivery to a living body that will provide a benefit;
selecting an a parasite appropriate for delivery of the identified fluid or biological material;
altering the selected parasite to have at least one functionality different from or unnatural compared to an unaltered parasite including a termination function capable of receiving a termination signal, wherein the termination signal results in the death or incapacitation of the altered parasitic organism; and
receiving data from the altered parasitic organism including or receiving or receiving data associated with the condition of the living body.

9. The method of claim 8, wherein receiving data comprises detecting a compound, nucleic acid or protein produced by the parasitic organism in response to a detected chemical or environmental condition within the living body or detecting a protein, nucleic acid or molecule manufactured by the parasitic organism for delivery to the living body.

10. The method of claim 8, wherein receiving data comprises detecting a protein, nucleic acid or molecule produced by the living body.

11. The method of claim 8, wherein the altered parasitic organism is capable of manufacturing a fluid or biological material comprising one selected from the group of a protein, a nucleic acid, and an organic compound.

12. The method of claim 11, further comprising sending an initiation signal to the altered parasitic organism, wherein the initiation signal results in the altered parasitic organism manufacturing a fluid or biological material or allows the altered parasitic organism to receive a fluid or biological material for delivery to the living body.

13. The method of claim 8, wherein receiving data associated with the condition of the living body comprises data indicative of a receptor binding event on the altered parasitic organism.

14. A system for evaluating and logging alterations in a parasite, comprising:
an alteration component for introducing an alteration into a parasite, wherein the alteration component has the capability to analyze the altered parasite;
an interface component in communication with the alteration component, wherein the interface component receives data from the alteration component associated with the condition of the parasite;
an operation component that analyzes received data associated with the condition of the parasite or provides instructions for delivery to the interface component and the alteration component, and logs the condition of the parasite.

15. The event logging system of claim 14, the parasitic is one species selected from the group comprising: mosquitoes, fleas, ticks, bed bugs (Cimicidae Cimex lectularius), midges (such as Ceratopogonidae), other blood sucking arthropods, annelids or leeches, nematodes such as Ascaris lumbricoides (roundworm which typically invades the gastrointestinal tract and lungs), pinworms such as Enterobius vermicularis (gastrointestinal tract, colon, fingertips), whipworms such as Trichuris trichiuria (gastrointestinal tract), flukes or trematodes such as Fasciola hepatica, Fasciolopsis buski (intestinal fluke) and schistosomes (liver and gallbladder), tapeworms or cestodes such as those from the genus Taenia (gastrointestinal tract), hookworms, heart worms, roundworms, lice (head, body, and pubic), and the like.

16. The event logging system of claim 14, wherein the alteration component alters the parasite to have a termination function, the termination function capable of receiving a termination signal and wherein the termination signal results in the death or reduced viability of the altered parasitic organism

17. The event logging system of claim 14, wherein the operation component generates data including selecting an alteration methodology to be used by the alteration component and delivers the data including an alteration methodology to the altering component.

18. The event logging system of claim 15, wherein the operation component that analyzes received data associated with the condition of the parasite analyzes the data to determine if the alteration methodology should be repeated, if a new alteration methodology is appropriate, or if the condition of the parasite is acceptable.

19. The event logging system of claim 14, wherein the parasite is altered to have the capability to produce a compound, nucleic acid or protein in response to a detected chemical or environmental condition within a living body.

20. The event logging system of claim 14, wherein the parasite is altered to have a receptor capable of binding to a compound, protein or nucleic acid present within a living body.
